(19) United States
(12) Patent Application Publication
(10) Pub. No.: US 2020/0352414 A1
(43) Pub. Date: Nov. 12, 2020

(54) INSERTING APPARATUS

(71) Applicant: OLYMPUS CORPORATION, Tokyo (JP)

(72) Inventors: Fumitoshi HAYAKAWA, Tokyo (JP); Akihiro KATO, Tokyo (JP); Kazuki MINAMIMOTO, Tokyo (JP)

(73) Assignee: OLYMPUS CORPORATION, Tokyo (JP)

(21) Appl. No.: 16/943,027

(22) Filed: Jul. 30, 2020

Related U.S. Application Data
(63) Continuation of application No. PCT/JP2018/032172, filed on Aug. 30, 2018.

(30) Foreign Application Priority Data
Jan. 31, 2018 (JP) 2018-015051

(51) Int. Cl. A61B 1/005

(52) U.S. Cl. CPC A61B 1/0055; A61B 1/0057; A61B 1/0052

(57) ABSTRACT

An inserting apparatus includes a base, a first recess, an opening forming surface, a movable member, a tabular lid member including a contact surface, the lid member closing an opening of the first recess, a second recess provided on the contact surface of the lid member, fixing resin for fixing the lid member to the base, and a third recess being a cutout recessed from a sidewall of at least one of the first recess and the second recess along a boundary between the opening fanning surface and the contact surface and including, on an inner surface, the boundary between the opening forming surface and the contact surface. The opening arming surface of the base and the contact surface of the second recess are provided at least on one side of both ends defining a moving range, the moving range being a range in which the movable member moves.

### CROSS REFERENCE TO RELATED APPLICATION

This application is a continuation application of PCT/JP2018/032172 filed on Aug. 30, 2018 and claims benefit of Japanese Application No. 2018-015051 filed in Japan on Jan. 31, 2018, the entire contents of which are incorporated herein by this reference.

### BACKGROUND OF THE INVENTION

### 1. Field of the Invention

The present invention relates to an inserting apparatus including an insertion section inserted into a subject and a movable member provided in the insertion section.

### 2. Description of the Related Art

As an inserting apparatus including an insertion section inserted into a subject such as an endoscope or a medical treatment instrument, there is a type including a movable member in the insertion section. For example, Japanese Patent Application Laid-Open Publication No. 2007-136044 discloses an endoscope including an erecting lever, which is a movable member, and an erecting stand in an insertion section.

In the endoscope disclosed in Japanese Patent Application Laid-Open Publication No. 2007-136044, the erecting lever is disposed in a lever housing section, which is a concave space formed in a distal end portion main body of the insertion section. In the endoscope disclosed in Japanese Patent Application Laid-Open Publication No. 2007-136044, the lever housing section is closed by a thin tabular cover fixed to the distal end portion main body by resin for bonding.

Since the inserting apparatus is repeatedly used, improvement of resistance against physical shocks is required for the inserting apparatus. Accordingly, for the inserting apparatus including the cover that closes the lever housing section disclosed in Japanese Patent Application Laid-Open Publication No. 2007-136044, a method of forming the cover as a thick member and improving strength of the cover is conceivable.

A reduction in a diameter of the insertion section is required for the inserting apparatus. Accordingly, for example, in the endoscope disclosed in Japanese Patent Application Laid-Open Publication No. 2007-136044, when a thick portion is provided in the cover that closes the lever housing section, a method a moving a bonding surface of the cover and the distal end portion main body to an inner side (a radial direction inner side) of the distal end portion main body and preventing the thickened cover from projecting to an outside is conceivable.

### SUMMARY OF THE INVENTION

An inserting apparatus according to an aspect of the present invention includes: a base provided in an insertion section inserted into a subject; a first recess recessed on a surface of the base; an opening forming surface formed around an opening of the first recess on the surface of the base; a movable member configured to move with respect to the base in the first recess; a lid member with a tabular shape including a contact surface in contact with the opening forming surface, the lid member closing the opening of the first recess; a second recess provided on the contact surface of the lid member, the second recess being configured to cover the movable member when the second recess is connected to the first recess in a state in which the lid member closes the opening; fixing resin for fixing the lid member to the base: and a third recess which is a cutout recessed from a sidewall of one or both of the first recess and the second recess along a boundary between the opening forming surface and the contact surface, the third recess including, on an inner surface, the boundary between the opening forming surface and the contact surface, wherein the opening forming surface of the base and the contact surface of the second recess are provided at least on one side of both ends defining a moving range, the moving range being a range in which the movable member moves.

### BRIEF DESCRIPTION OF THE DRAWINGS

FIG. 1 is a diagram showing a schematic configuration of a side view-type endoscope;

FIG. 2 is a perspective view of a distal end portion of an insertion section;

FIG. 3 is a perspective view showing a distal end cover and a distal end member in a separated state;

FIG. 4 is a diagram showing a left side surface of the distal end portion in a state in which the distal end cover is not attached;

FIG. 5 is a V-V sectional view of FIG. 4;

FIG. 6 is a VI-VI sectional view of FIG. 4;

FIG. 7 is an exploded perspective view of the distal end member;

FIG. 8 is a VIII-VIII sectional view of FIG. 5;

FIG. 9 is a partial sectional view showing a right side surface of the distal end member;

FIG. 10 is a diagram showing the right side surface of the distal end member in a state in which a lid member is detached;

FIG. 11 is an XI-XI sectional view of FIG. 4;

FIG. 12 is an enlarged view of a portion surrounded by a circle drawn by a broken line in FIG. 6;

FIG. 13 is a diagram showing a first modification of a third recess portion; and

FIG. 14 is a diagram showing a second modification of the third recess portion.

### DETAILED DESCRIPTION OF THE PREFERRED EMBODIMENT

A preferred embodiment of the present invention is explained below with reference to the drawings. Note that, in respective figures used for the following explanation, scales are differentiated for each of components in order to show the respective components in recognizable sizes on the drawings. The present invention is not limited to only quantities of the components, shapes of the components, ratios of the sizes of the components, and relative positional relations among the respective components described in these figures.

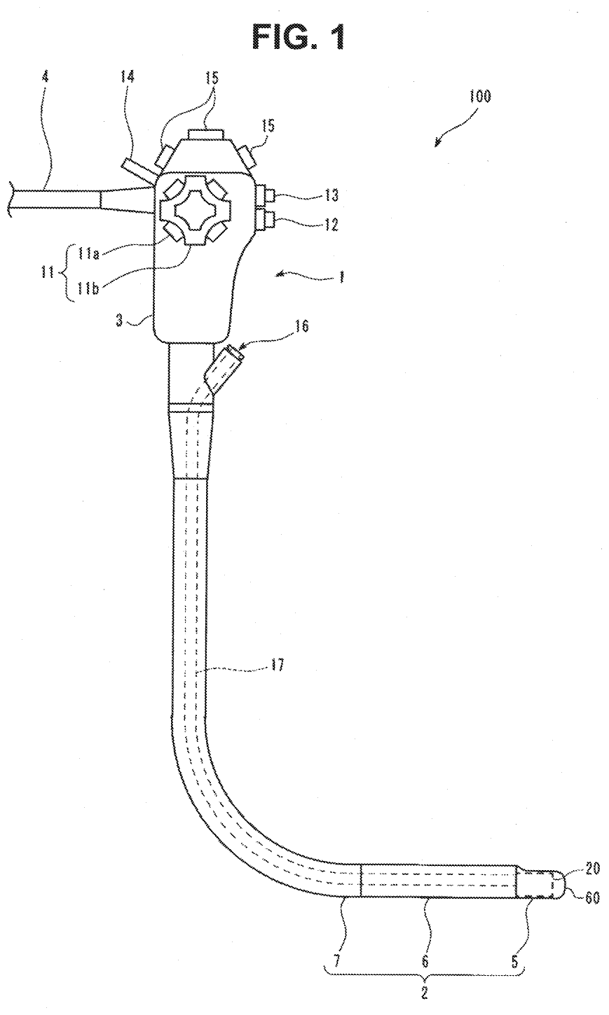

FIG. 1 is a diagram showing a schematic configuration of an inserting apparatus 100. The inserting apparatus 100 in the present embodiment includes an inserting apparatus main body 1 and a distal end cover 60. In the present embodiment, as an example, the inserting apparatus 100 is an endoscope including an insertion section 2 inserted into a human body, which is a subject, and is, more specifically, a side view-type endoscope for duodenum.

The inserting apparatus main body 1 includes the insertion section 2 inserted into the subject, an operation section 3 provided on a proximal end side of the insertion section 2, and a universal cord 4 extended from the operation section 3.

In the operation section 3, a bending operation apparatus 11, an air/water feeding button 12, a suction button 13, a raising base operation lever 14, and an operation switch 15 are provided. The operation switch 15 is an electronic switch for operating an image pickup apparatus 42 (not shown in FIG. 1) provided in the insertion section 2.

In the operation section 3, a treatment instrument insertion port 16 for introducing a not-shown treatment instrument into a body is provided. A proximal end side of a channel tube 17 is connected to the treatment instrument insertion port 16. A distal end side of the channel tube 17 is opened at a distal end portion 5 of the insertion section 2.

The insertion section 2 is configured by concatenating the distal end portion 5 disposed at a distal end, a bendable bending section 6 disposed on a proximal end side of the distal end portion 5, and a flexible tube section 7 having flexibility that connects a proximal end side of the bending section 6 and the operation section 3. The distal end cover 60 is attached to the distal end portion 5. Details of configurations of the distal end portion 5 and the distal end cover 60 are explained below.

The bending section 6 bends in an upward direction and a downward direction according to rotation of an upper and lower bending knob 11a of the bending operation apparatus 11 provided in the operation section 3 and bends in a left direction or a right direction according to rotation of a left and right bending knob 11b.

A raising base operation wire 18 (not shown in FIG. 1) is inserted through the insertion section 2. The raising base operation wire 18 advances and retracts in a longitudinal direction according to swinging of the raising base operation lever 14. In other words, the raising base operation lever 14 is an operation member for a user to operate an apparatus that pushes and pulls the raising base operation wire 18 inserted through the insertion section 2. A distal end of the raising base operation wire 18 is connected to a movable member 51 (not shown in FIG. 1) explained below provided in the distal end portion 5.

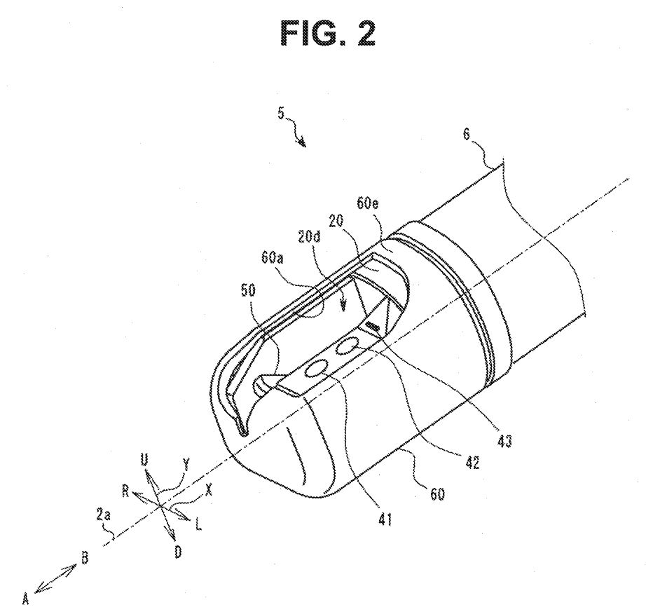

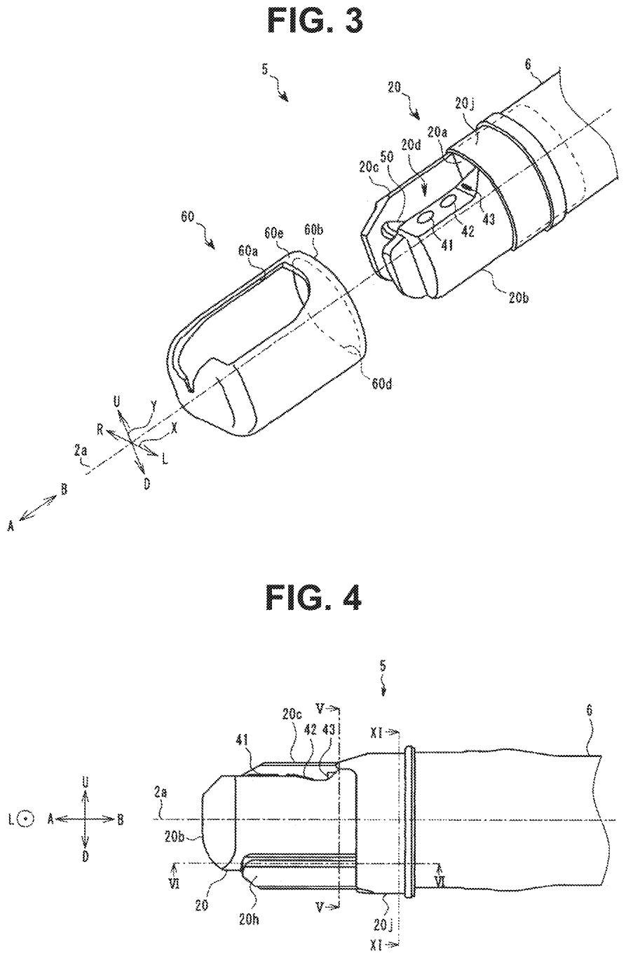

FIG. 2 is a perspective view of the distal end portion 5. As shown in FIG. 2, the distal end cover 60 is attached to the distal end portion 5. The distal end cover 60 is a sheath-like member that covers a predetermined outer surface of the distal end portion 5. The distal end cover 60 is detachably attachable to the distal end portion 5. FIG. 3 is a perspective view showing the distal end cover 60 and the distal end portion 5 in a separated state.

In the present embodiment, as an example, the distal end cover 60 is formed of resin having low elasticity compared with rubber and the like and easily plastically deformed or fractured among kinds of resin such as polyethylene and polypropylene. In the distal end cover 60 in the present embodiment, irreversible deformation and fracture occur when the distal end cover 60 is detached from the distal end portion 5 after being attached to the distal end portion 5 and cannot be reused. In FIG. 3, the distal end cover 60 in a state in which the distal end cover 60 is not attached to the distal end portion 5 at all (an unused state) is shown.

A configuration of the distal end portion 5 is explained. Note that, in the following explanation, an axis in the longitudinal direction of the elongated insertion section 2 is referred to as a longitudinal axis 2a. A direction toward a distal end side of the insertion section 2 along the longitudinal axis 2a is referred to as distal end direction A. The opposite direction of the distal end direction A is referred to as proximal end direction B. Two linear axes orthogonal to each other on a plane orthogonal to the longitudinal axis 2a are defined as an X axis and a Y axis. A direction toward one side in the X direction is referred to as right direction R. The opposite direction of the right direction R is referred to as left direction L. A direction toward one side in the Y axis is referred to as upward direction U. The opposite direction of the upward direction U is referred to as downward direction D. The X axis and the Y axis are substantially parallel to a bending direction of the bending section 6. In the present embodiment, as an example, when viewed from the proximal end side toward the distal end side in the longitudinal axis 2a and when the X axis is horizontal, it is assumed that a right side is the right direction R and an upper side is the upward direction U.

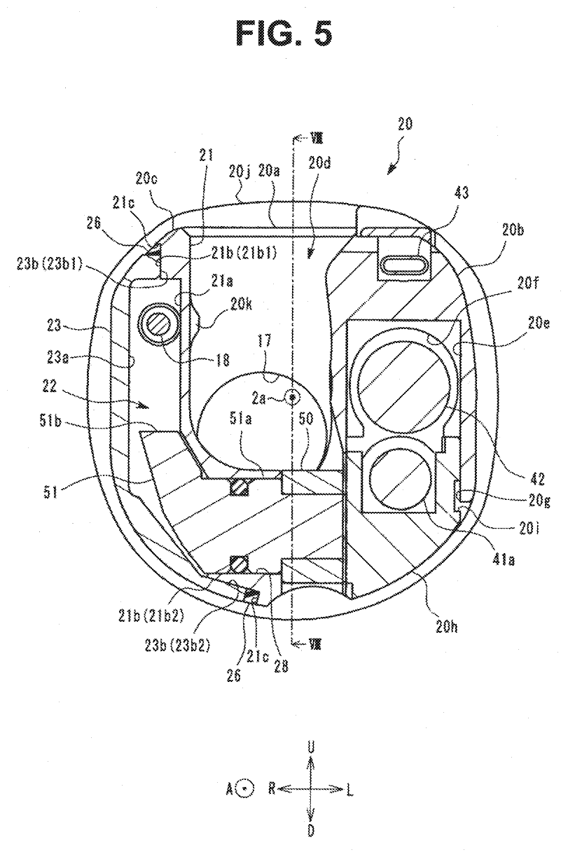

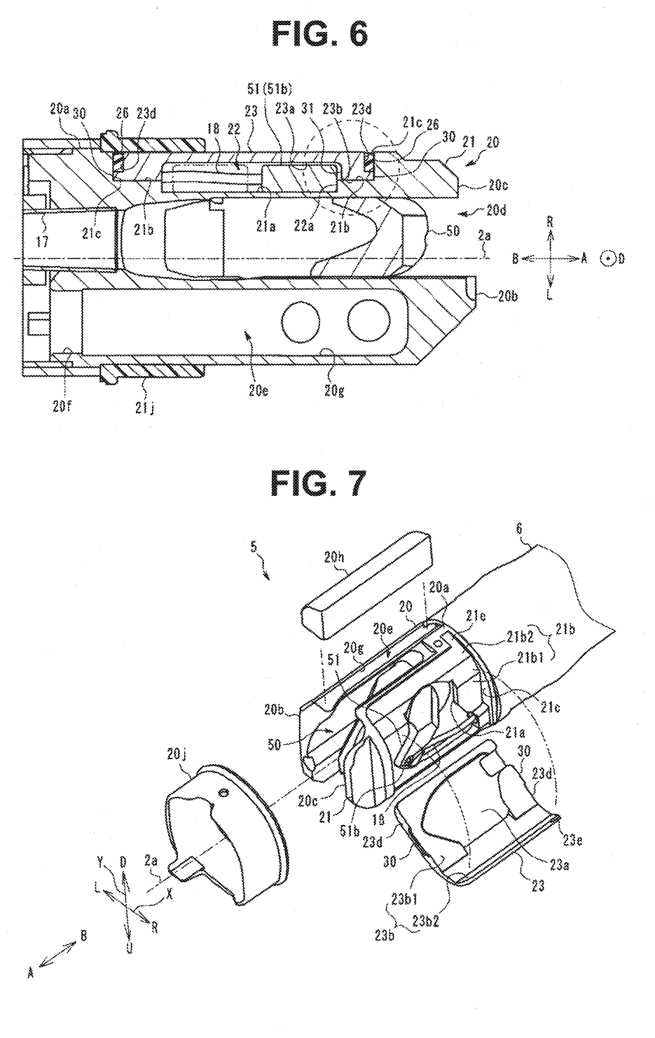

FIG. 4 is a diagram showing a left side surface of the distal end portion 5 in a state in which the distal end cover 60 is not attached. FIG. 5 is a V-V sectional view of FIG. 4. FIG. 6 is a VI-VI sectional view of FIG. 4. FIG. 7 is an exploded perspective view of a distal end member 20.

As shown in FIG. 3, the distal end portion 5 includes the distal end member 20 and an insulating section 20j made of resin or ceramic having electric insulation. The distal end member 20 includes a base portion 20a fixed to a distal end of the bending section 6, a first arm section 20b and a second arm section 20c, which are a pair of arm sections projecting in the distal end direction A from the base portion 20a, and a raising base housing space 20d, which is a space formed between the first arm section 20b and the second arm section 20c. An outer shape of the base portion 20a is a substantially columnar shape. An outer circumference of the base portion 20a is covered by the insulating section 20j having an annular shape.

The first arm section 20b and the second arm section 20c are disposed such that the raising base housing space 20d, which is the space formed between the first arm section 20b and the second arm section 20c, is opened in three directions of the upward direction U, the downward direction D, and the distal end direction A. In other words, the first arm section 20b and the second arm section 20c are arrayed in a direction along the X axis with the raising base housing space 20d in between. In the present embodiment, as an example, the first arm section 20b is disposed on the left direction L side of the raising base housing space 20d and the second arm section 20c is disposed on the right direction R side of the raising base housing space 20d.

An illumination lens 41, an image pickup apparatus 42, and a cleaning nozzle 43 are disposed on an upper surface facing the upward direction U in an outer circumferential surface of the first arm section 20b. The illumination lens 41 is a lens for emitting illumination light toward an object of the image pickup apparatus 42. The illumination light is guided from a light emitting apparatus to the illumination lens 41 via an optical fiber cable 41a inserted into the insertion section 2. The light emitting apparatus may be disposed in the inserting apparatus 100 or may be disposed in an external apparatus connected to the inserting apparatus 100.

A visual field of the image pickup apparatus 42 is generally centered on the upward direction U. In other words, the image pickup apparatus 42 catches a side direction of the insertion section 2 in the visual field. The cleaning nozzle 43 is a part that jets fluid toward the illumination lens 41 and the image pickup apparatus 42.

As shown in FIG. 5, an image pickup apparatus housing space 20e is formed on an inside of the first arm section 20b. A distal end portion of the optical fiber cable 41a and the image pickup apparatus 42 are disposed in the image pickup apparatus housing space 20e.

The image pickup apparatus housing space 20e communicates with an internal space of the bending section 6 via a through-hole 20f that pierces through the base portion 20a. The distal end portion of the optical fiber cable 41a and the image pickup apparatus 42 are inserted into the image pickup apparatus housing space 20e through the through-hole 20f.

The image pickup apparatus housing space 20e includes an opening section 20g opened on a lower surface facing the downward direction D of the first area section 20b as shown in FIG. 7. The opening section 20g is closed by a plug body 20h fit into the opening section 20g. The plug body 20h is fixed in the opening section 20g by an adhesive. As shown in FIG. 5, a claw 20i for catching a tool, for example, when the plug body 20h is pulled out from the opening section 20g during maintenance of the inserting apparatus 100 is formed in the plug body 20h.

As explained in detail below, a housing space 22, in which the movable member 51 is disposed, is provided in the second arm section 20c. The movable member 51 is a member that transmits a movement of the raising base operation wire 18 to a raising base (forceps elevator) 50.

The distal end cover 60 is a sheath-like member closed on the distal end direction A side and opened on the proximal end direction B side. An opening provided on the proximal end direction B side of the distal end cover 60 is referred to as an insertion port 60d. When the distal end cover 60 is attached to the distal end portion 5, the distal end portion S is inserted into the distal end cover 60 through the insertion port 60d.

The distal end cover 60 includes an opening section 60a that exposes the raising base housing space 20d in only the upward direction U in a state in which the distal end cover 60 is attached to the distal end portion 5. In a state in which the distal end cover 60 is attached to the distal end member 20, the illumination lens 41, the image pickup apparatus 42, and the cleaning nozzle 43 are also exposed in the upward direction U via the opening section 60a.

On an outer surface of the distal end cover 60, the opening section 60a is not connected to the insertion port 60d. Accordingly, an annular section 60e, an entire circumference of which is annularly connected around the longitudinal axis 2a, is formed at a proximal end 60b of the distal end cover 60. In the state in which the distal end cover 60 is attached to the distal end member 20, the annular section 60e fits with an outer circumference of the insulating section 20j.

Configurations of the housing space 22 provided in the second arm section 20c of the distal end member 20 and the movable member 51 disposed in the housing space 22 are explained.

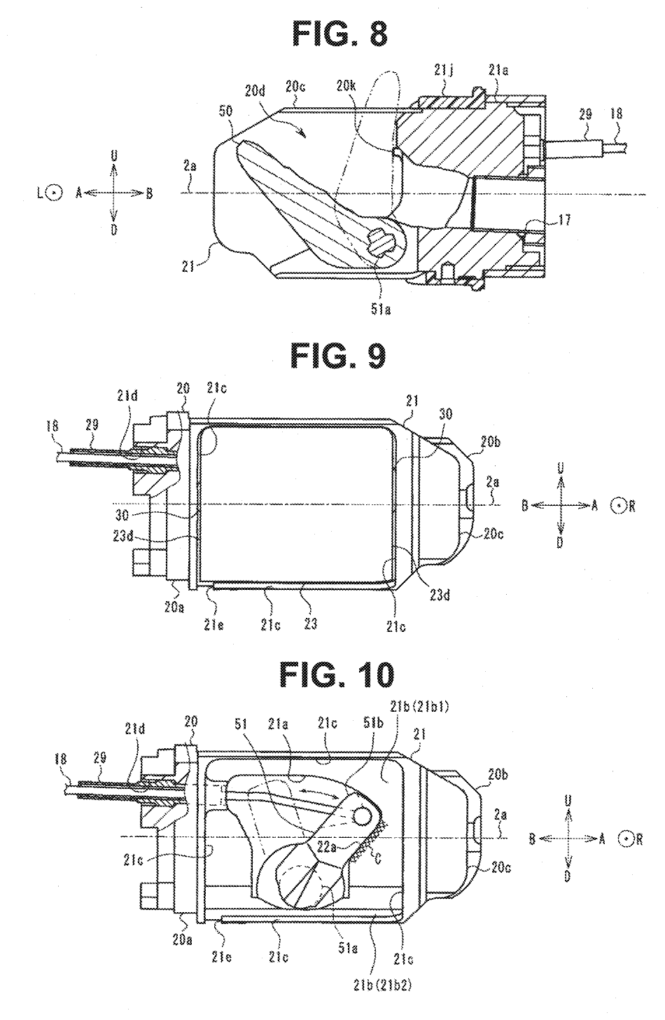

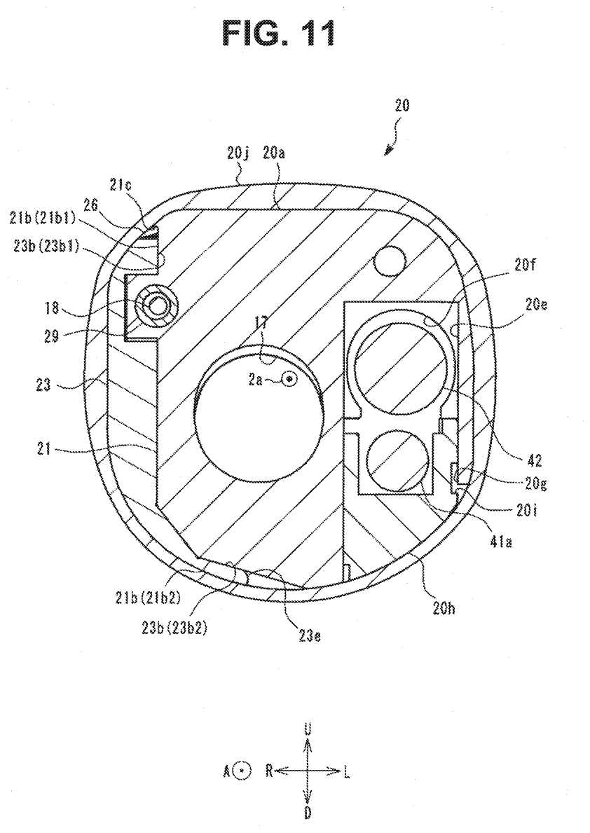

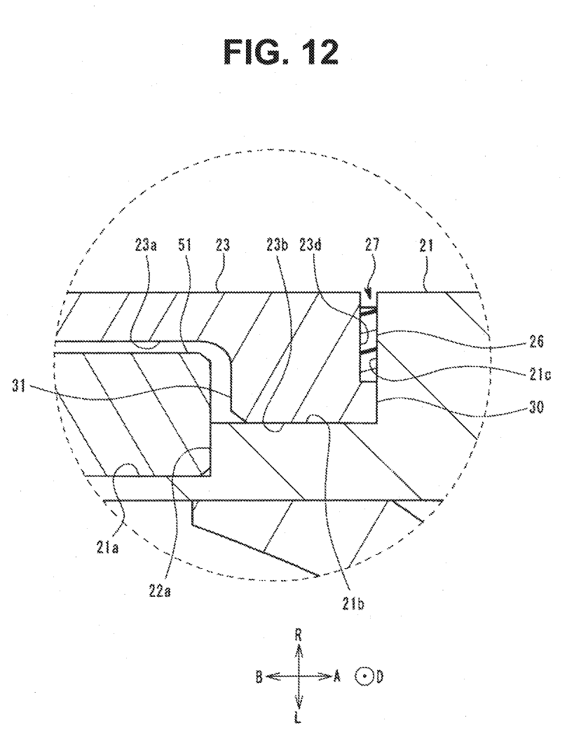

FIG. 8 is a VIII-VIII sectional view of FIG. 5. FIG. 9 is a partial sectional view showing a right side surface of the distal end member 20. FIG. 10 is a diagram showing the right side surface of the distal end member 20 in a state in which a lid member 23 is detached. FIG. 11 is an XI-XI sectional view of FIG. 4. FIG. 12 is an enlarged view of a portion surrounded by a circle drawn by a broken line in FIG. 6.

As shown in FIG. 5, FIG. 6, and FIG. 7, the second arm section 20c is configured by a base section 21 integral with the distal end member 20 and the lid member 23 bonded and fixed to the base section 21 by fixing resin 26. In the present embodiment, the lid member 23 is fixed to a left side surface of the base section 21. The housing space 22 is configured by a first recess portion 21a and a second recess portion 23a respectively recessed on surfaces facing each other of the base section 21 and the lid member 23.

More specifically, the base section 21 includes a first recess portion 21a, an opening forming surface 21b, and a wall surface section 21c. The first recess portion 21a is opened in the opening forming surface 21b in a surface of the base section 21. In other words, the opening forming surface 21b is a surface formed to surround a circumference of an opening of the first recess portion 21a.

A shape of the opening forming surface 21b is not particularly limited and, for example, may be formed by a single plane, may be formed by a plurality of planes, or may be formed by a curved surface.

In the present embodiment, as an example, as shown in FIG. 5 and FIG. 11, the opening forming surface 21b includes a pair of planes 21b1 and 21b2 substantially parallel to the longitudinal axis 2a and disposed at angles at which the pair of planes 21b1 and 21b2 cross each other. The pair of planes 21b1 and 21b2 face different directions on a surface of the columnar base section 21 such that a crossing line of the pair of planes 21b1 and 21b2 is formed as a ridgeline convex to an outer side.

The opening forming surface 21b including the pair of planes 21b1 and 21b2 is formed in a shape bent in a ridge shape in a cross section orthogonal to the longitudinal axis 2a.

The wall surface section 21c is erected toward an outer side of the base section 21 from the opening forming surface 21b. The wall surface section 21c is explained below.

The lid member 23 is a tabular member and includes a second recess portion 23a, a contact surface 23b, and a protrusion 30. The contact surface 23b is a surface in contact with the opening forming surface 21b. The contact surface 23b is larger than the opening of the first recess portion 21a and is in contact with the opening forming surface 21b over an entire circumference of the opening of the first recess portion 21a. Therefore, in a state in which the contact surface 23b is in contact with the opening forming surface 21b, the opening of the first recess portion 21a is closed by the lid member 23.

Since an outer circumferential portion of the second recess portion 23a, that is, a portion near a side surface 23d is thicker than a center portion where the second recess portion 23a is formed, the lid member 23 has higher strength compared with a plate having thickness equivalent to thickness in the center portion.

The contact surface 23b has a shape adhering to the opening forming surface 21b in order to close the first recess portion 21a. Specifically, in the present embodiment, since the opening forming surface 21b has the shape bent in the ridge shape in the cross section parallel to the longitudinal axis 2a, the contact surface 23b has a shape bent in a trough shape according to the bending of the opening forming surface 21b. In other words, the contact surface 23b includes a pair of planes 23b1 and 23b2 parallel to the pair of planes 21b1 and 21b2 of the opening forming surface 21b.

Since the opening forming surface 21b on the base section 21 side has the ridge shape and the contact surface 23b on the lid member 23 side has the trough shape in this way, the lid member 23 is positioned in a circumferential direction around the longitudinal axis 2a with respect to the base section 21 by bringing the contact surface 23b into contact with the opening forming surface 21b. In the present embodiment, the positioning of the lid member 23 in the circumferential direction around the longitudinal axis 2a with respect to the base section 21 is positioning in the upward direction U and the downward direction D generally along the Y axis.

The second recess portion 23a is opened in the contact surface 23b. In other words, a circumference of an opening of the second recess portion 23a is surrounded by the contact surface 23b. The second recess portion 23a is connected to the first recess portion 21a in a state in which the lid member 23 closes the opening of the first recess portion 21a.

As explained above, the housing space 22 is formed by bonding the base section 21 and the lid member 23 such that the first recess portion 21a provided in the base section 21 and the second recess portion 23a provided in the lid member 23 face each other. Therefore, on a sidewall surface of the housing space 22, a boundary line between the base section 21 and the lid member 23 is present in a position separated from respective bottom surfaces of the first recess portion 21a and the second recess portion 23a.

The sidewall surface of the housing space 22 is a surface excluding the bottom surfaces of the first recess portion 21a and the second recess portion 23a in an inner surface of the housing space 22. In other words, the sidewall surface of the housing space 22 is formed by sidewalls of the first recess portion 21a and the second recess portion 23a. The boundary line is a mating surface of the base section 21 and the lid member 23, that is, the opening forming surface 21b and the contact surface 23b appearing on the sidewall surface of the housing space 22. In the present embodiment, the mating surface of the base section 21 and the lid member 23 is substantially parallel to the longitudinal axis 2a.

The wall surface section 21e provided in the base section 21 and the protrusion 30 provided in the lid member 23 are explained.

The wall surface section 21c is erected toward the outer side of the base section 21 from the opening forming surface 21b. The wall surface section 21c has a surface facing at least a part of the side surface 23d of the lid member 23 in a state in which the contact surface 23b is in contact with the opening forming surface 21b.

The wall surface section 21c in the present embodiment is disposed to surround a circumference of the lid member 23 at a predetermined distance apart from the side surface 23d. In the present embodiment, the wall surface section 21c is a sidewall surface of a recess portion formed on a surface of the base section 21 and having a shape, on an inner side in which the lid member 23 is fit. The opening forming surface 21b corresponds to a bottom surface of the recess portion.

The protrusion 30 projects toward the wall surface section 21c from a part of the side surface 23d of the lid member 23. As shown in FIG. 6 and FIG. 12, the protrusion 30 has thickness smaller than thickness in the side surface 23d of the lid member 23. The protrusion 30 is in contact with the wall surface section 21c, whereby the side surface 23d in a portion where the protrusion 30 is provided is prevented from adhering to the wall surface section 21c. A gap 27 having predetermined width is farmed between the side surface 23d and the wall surface section 21c.

In the present embodiment, as an example, the lid member 23 generally has a rectangular shape. The side surface 23d of the lid member 23 includes a pair of surfaces substantially parallel to the longitudinal axis 2a and a pair of surfaces substantially orthogonal to the longitudinal axis 2a. The lid member 23 includes a plurality of protrusions 30 projecting in both directions (the distal end direction A and the proximal end direction B) along the longitudinal axis 2a from a pair of side surfaces 23d substantially parallel to the longitudinal axis 2a.

The wall surface section 21c provided in the base section 21 is disposed to hold the lid member 23 in both directions along the longitudinal axis 2a. Therefore, in the present embodiment, the protrusion 30 is in contact with the wall surface section 21c, whereby the lid member 23 is positioned in the longitudinal axis 2a direction with respect to the base section 21.

The lid member 23 is positioned in a direction (a circumferential direction) orthogonal to the longitudinal axis 2a with respect to the base section 21 by bringing the contact surface 23b into contact with the opening forming surface 21b as explained above. Therefore, in the present embodiment, when the lid member 23 is bonded to the base section 21, the lid member 23 can be positioned on the surface of the base section 21 by bringing the contact surface 23b into contact with the opening forming surface 21b and bringing the protrusion 30 into contact with the wall surface section 21c.

At this time, the gap 27 having a groove shape is formed between the lid member 23 and the wall surface section 21c surrounding the circumference of the lid member 23. Since the protrusion 30 is thinner than the wall surface section 21c, the gap 27 formed around the lid member 23 continues without being cut along an outer circumference of the contact surface 23b of the lid member 23.

Fixing resin 26 before curing is poured into the gap 27 formed between the lid member 23 and the wall surface section 21c and, thereafter, the fixing resin 26 is cured, whereby the lid member 23 is bonded and fixed to the base section 21. In the present embodiment, since the fixing resin 26 can be closely disposed without being cut along the outer circumference of the contact surface 23b of the lid member 23, it is possible to surely and firmly fix the lid member 23 to the base section 21.

In the present embodiment, as shown in FIG. 7 and FIG. 11, in a part of a corner where the contact surface 23b of the lid member 23 and the side surface 23d cross, a chamfered section 23e formed by cutting out the corner is formed. A cutout 21e is formed in the wall surface section 21c facing the side surface 23d on which the chamfered section 23e is formed. For example, when the lid member 23 is detached from the base section 21 during maintenance of the inserting apparatus 100, a tool can be inserted into a gap between the chamfered section 23e and the opening forming surface 21b.

The movable member 51 disposed in the housing space 22 is explained.

As shown in FIG. 5, FIG. 6, and FIG. 10, the movable member 51 is supported by the base section 21 to be movable with respect to the base section 21 in the first recess portion 21a. Specifically, a bearing 28 piercing through from a bottom surface of the first recess portion 21a to the raising base housing space 20d is formed in the base section 21. The bearing 28 turnably supports a shaft section 51a fixed to the movable member 51. The movable member 51 includes a lever 51b extending from the shaft section 51a in a direction orthogonal to the shaft section 51a in the first recess portion 21a. Therefore, the lever 51b swings around a rotation axis of the shaft section 51a in the first recess portion 21a.

In the present embodiment, the rotation axis of the movable member 51 is substantially parallel to the X axis. Therefore, the lever 51b moves in a direction generally along the longitudinal axis 2a in the first recess portion 21a (the housing space 22).

A distal end of the raising base operation wire 18 is connected to the lever 51b in a position separated from the shaft section 51a by a predetermined distance. The raising base operation wire 18 is inserted into a through-hole 21d that pierces through the base section 21 and the base portion 20a from a side surface of the first recess portion 21a in the proximal end direction B. Note that, in the present embodiment, a guide pipe 29 that guides the raising base operation wire 18 is inserted into the through-hole 21d. The guide pipe 29 is a rigid tubular member. The raising base operation wire 18 is inserted through the guide pipe 29.

As explained above, the raising base operation wire 18 advances and retracts in a direction along the longitudinal axis 2a according to the swinging of the raising base operation lever 14. Therefore, the lever 51b swings around the rotation axis according to the swinging of the raising base operation lever 14.

The shaft section 51a extends into the raising base housing space 20d. The raising base 50 is fixed to a portion of the shaft section 51a extending into the raising base housing space 20d. Therefore, as shown in FIG. 8, the raising base 50 swings around the rotation axis of the shaft section 51a together with the movable member 51. In other words, the raising base 50 swings around the rotation axis according to the swinging of the raising base operation lever 14.

The raising base 50 is a tongue-like member extending in one direction from the shaft section 51a. The channel tube 17 is opened on the proximal end direction B side of the raising base 50 of the raising base housing space 20d. An angle of the treatment instrument projecting from the channel tube 17 changes according to the swinging of the raising base 50.

A part of the lever 51b of the movable member 51 further projects than the opening forming surface 21b from the opening of the first recess portion 21a. In other words, a part of the movable member 51 projects to an inside of the second recess portion 23a formed in the lid member 23.

In other words, in the present embodiment, the movable member 51 moving in the housing space 22 is disposed to move substantially in parallel to the mating surface of the base section 21 and the lid member 23. A part of the movable member 51 projects from the first recess portion 21a to the inside of the second recess portion 23a beyond the opening forming surface 21b (the contact surface 23b), which is the mating surface of the base section 21 and the lid member 23. Therefore, when viewed from a direction parallel to the mating surface of the base section 21 and the lid member 23 (for example, when viewed from a visual line direction in FIG. 6), the movable member 51 overlaps the mating surface of the base section 21 and the lid member 23.

A configuration for defining a moving range of the movable member 51 in the housing space 22 is now explained.

At least one of both ends in the moving range of the movable member 51 in the housing space 22 is defined by a position where the movable member 51 is in contact with a restricting portion 22a, which is a part of a sidewall of the housing space 22.

Specifically, in the present embodiment, an end on the distal end direction A side in the moving range of the movable member 51 is a position where the movable member 51 is in contact with the restricting portion 22a provided in a part of a sidewall of the first recess portion 21a. FIG. 6 and FIG. 10 show a state in which the movable member 51 is located at the end on the distal end direction A side of the moving range and is in contact with the restricting portion 22a.

As explained above, when viewed from the direction parallel to the mating surface of the base section 21 and the lid member 23, the movable member 51 moves in the housing space 22 in a state in which the movable member 51 overlaps the mating surface of the base section 21 and the lid member 23. Accordingly, the restricting portion 22a provided on the sidewall of the first recess portion 21a projects to the proximal end direction B side relative to a sidewall of the second recess portion 23a in order to come into contact with, earlier than the sidewall of the second recess portion 23a, the movable member 51 moving in the distal end direction A in the housing space 22.

In other words, an opening width in the longitudinal axis 2a direction of the second recess portion 23a is larger than an opening width of the first recess portion 21a. The sidewall of the second recess portion 23a is separated in the longitudinal axis 2a direction from the movable member 51 in a contact state with the restricting portion 22a.

In other words, a cutout recessed from the sidewall of one or both of the first recess portion 21a and the second recess portion 23a along a boundary between the opening forming surface 21b and the contact surface 23b (the mating surface of the base section 21 and the lid member 23), the cutout being a third recess portion 31, on an inner surface of which the mating surface appears, is provided on the sidewall of the housing space 22 in the present embodiment.

In the present embodiment, as an example, the third recess portion 31 is formed by cutting out only the sidewall of the second recess portion 23a to an outer side of the housing space 22 along the mating surface relative to the sidewall of the first recess portion 21a. By cutting out only the sidewall of the second recess portion 23a, in the third recess portion 31, a part of the opening forming surface 21b is exposed in the housing space 22.

The third recess portion 31 may be provided over an entire circumference of the sidewall of the housing space 22 or may be provided in only a region to which the movable member 51 comes close such as a vicinity of the restricting portion 22a. In the present embodiment, the third recess portion 31 is provided at least in the vicinity of the restricting portion 22a. In FIG. 10, a part where the third recess portion 31 is formed in the present embodiment is indicated by hatching marked with a sign C.

By providing, in the base section 21, which is the same member, the bearing 28 that supports the movable member 51 and the restricting portion 22a that defines the moving range of the movable member 51 as in the present embodiment, it is possible to improve accuracy of the moving range of the movable member 51. Note that the restricting portion 22a may be provided in the lid member 23 (the sidewall of the second recess portion 23a).

On the other hand, an end on the proximal end direction B side in the moving range of the movable member 51 is a position where the raising base 50 fixed to the movable member 51 is in contact with a stopper 20k that projects into the raising base housing space 20d. The stopper 20k is shown in FIG. 5 and FIG. 8. FIG. 8 shows a state in which the raising base 50 is in contact with the stopper 20k. By providing the stopper 20k, which is in direct contact with the raising base 50, in the raising base housing space 20d, it is possible to position the raising base 50 irrespective of fluctuation in fixing positions of the movable member 51 and the raising base 50.

As explained above, the inserting apparatus 100 in the present embodiment includes, in the insertion section 2 inserted into the subject, the housing space 22 for housing the movable member 51. The housing space 22 is formed by the first recess portion 21a formed in the base section 21 provided in the insertion section 2 and the second recess portion 23a formed in the lid member 23 bonded and fixed to the base section 21.

In the present embodiment, when viewed in the direction parallel to the mating surface of the base section 21 and the lid member 23, the movable member 51 moves in the housing space 22 in the state in which the movable member 51 overlaps the mating surface of the base section 21 and the lid member 23.

The fixing resin 26 for bonding and fixing the base section 21 and the lid member 23 is likely to flow into the housing space 22 from the mating surface at a stage before the fixing resin 26 is cured. However, in the present embodiment, since the third recess portion 31, which is the cutout provided along the mating surface in the housing space 22, is formed as explained above, the fixing resin 26 flowing into the housing space 22 flows to stay in the third recess portion 31. Accordingly, in the present embodiment, it is possible to prevent the fixing resin 26 from interfering with the movable member 51 in the housing space 22.

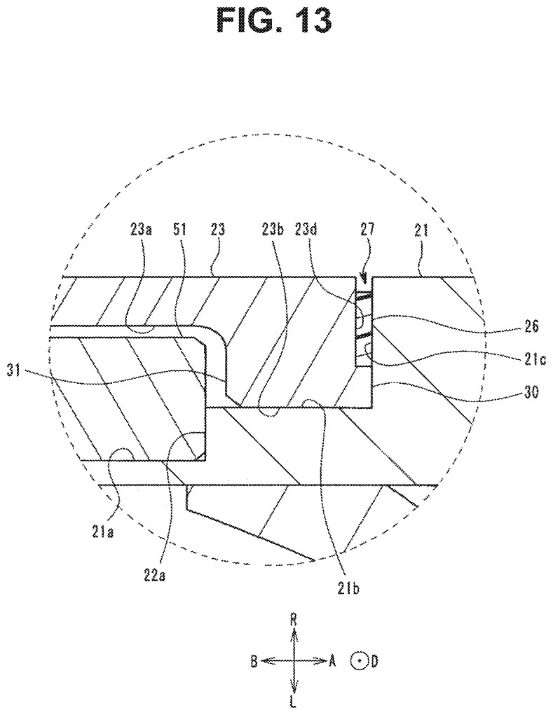

Note that, in the embodiment explained above, the third recess portion 31 is formed by cutting out only the sidewall of the second recess portion 23a of the lid member 23. However, a configuration of the third recess portion 31 is not limited to this. For example, as indicated by a first modification shown in FIG. 13, the third recess portion 31 may be formed by cutting out only the sidewall of the first recess portion 21a of the base section 21. In this case, the restricting portion 22a is formed by at least a part of the sidewall of the second recess portion 23a of the lid member 23.

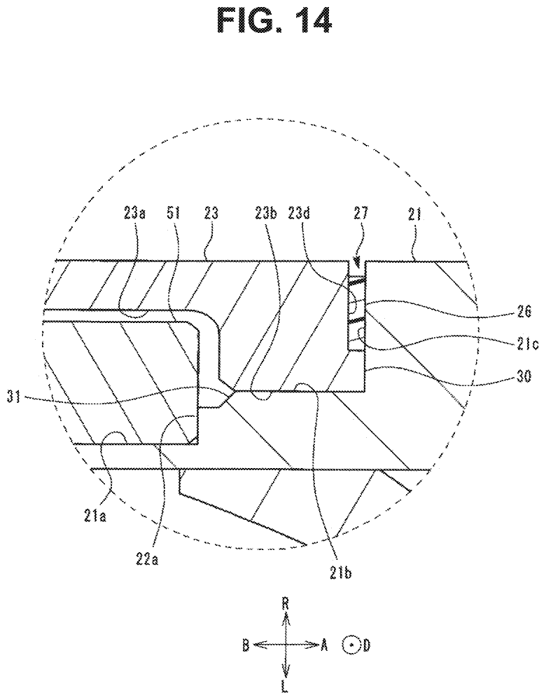

For example, as indicated by a second modification shown in FIG. 14, the third recess portion 31 may be formed by cutting out both of the sidewall of the first recess portion 21a of the base section 21 and the sidewall of the second recess portion 23a of the lid member 23. In both of the first and second modifications, the same effects as the effects in the embodiment explained above can be obtained.

The present invention is not limited to the embodiment explained above and can be changed as appropriate in a range not departing from the gist or the spirit of the invention read from the claims and the entire specification. An inserting apparatus involving such a change is also included in the technical scope of the present invention.

what is claimed is:

1. An inserting apparatus comprising:
a base provided in an insertion section inserted into a subject;
a first recess recessed on a surface of the base;
an opening forming surface formed around an opening of the first recess on the surface of the base;
a movable member configured to move with respect to the base in the first recess;
a lid member with a tabular shape including a contact surface in contact with the opening forming surface, the lid member closing the opening of the first recess;
a second recess provided on the contact surface of the lid member, the second recess being configured to cover the movable member when the second recess is connected to the first recess in a state in which the lid member closes the opening;
fixing resin for fixing the lid member to the base; and
a third recess which is a cutout recessed from a sidewall of one or both of the first recess and the second recess along a boundary between the opening forming surface and the contact surface, the third recess including, on an inner surface, the boundary between the opening forming surface and the contact surface, wherein
the opening forming surface of the base and the contact surface of the second recess are provided at least on one side of both ends defining a moving range, the moving range being a range in which the movable member moves.

2. The inserting apparatus according to claim 1, wherein at least one of both ends of the moving range of the movable member is a restricting portion provided on the sidewall of the first recess or the second recess, the movable member coming into contact with the restricting portion.

3. The inserting apparatus according to claim 2, wherein
the restricting portion is formed on the sidewall of the first recess, and
the third recess is formed by cutting out the sidewall of the second recess to expose the opening forming surface in a housing space.

4. The inserting apparatus according to claim 1, wherein the third recess is formed by cutting out the sidewall of the second recess to expose the opening forming surface in a housing space.

5. The inserting apparatus according to claim 1, wherein the inserting apparatus is an endoscope.

6. The inserting apparatus according to claim 1, wherein
the opening forming surface is formed in a surface shape in a longitudinal axis direction of the insertion section on a side surface of the base, and
the movable member moves within the moving range in the longitudinal axis direction with respect to the base.

7. The inserting apparatus according to claim 1, wherein the opening forming surface is formed to surround a circumference of an opening of the first recess.

8. The inserting apparatus according to claim 2, wherein the third recess is disposed in a vicinity of the restricting portion.

9. The inserting apparatus according to claim 8, wherein the vicinity of the restricting portion is a position adjacent to the restricting portion and is a position beyond both ends of the moving range.
